(19) United States
(12) Patent Application Publication
(10) Pub. No.: US 2025/0319466 A1
(43) Pub. Date: Oct. 16, 2025

(54) ACOUSTOPHORETIC ANALYZATION DEVICES AND METHODS

(71) Applicant: Siemens Healthcare Diagnostics Inc., Tarrytown, NY (US)

(72) Inventors: Jeffrey Jasperse, Newton, MA (US); Peter Paulicka, Forchheim (DE); Michael Palombo, South Boston, MA (US); Ramona Seliger, Wiesenttal (DE)

(73) Assignee: Siemens Healthcare Diagnostics Inc., Tarrytown, NY (US)

(21) Appl. No.: 18/873,257

(22) PCT Filed: Oct. 21, 2022
(86) PCT No.: PCT/US2022/078504

§ 371 Date: Dec. 9, 2024

Related U.S. Application Data
(60) Provisional application No. 63/367,347, filed on Jun. 30, 2022.

(51) Int. Cl. B01L 3/00; C12N 1/06

(52) U.S. Cl. CPC B01L 3/50273; B01L 3/502715; B01L 2200/10; B01L 2300/0816; B01L 2400/0439; C12N 1/06

(57) ABSTRACT

Fluid analyzation devices, methods, and systems are disclosed including an analyzation device comprising a sample vessel having an outer surface, a microchannel within the confines of the outer surface, a first port extending through the outer surface to the microchannel, and a second port extending through the outer surface to the microchannel; and an piezo transducer bonded to the outer surface of the sample vessel to form a monolithic structure, the piezo transducer configured to emit ultrasonic acoustic waves having a first frequency, a second frequency, and a third frequency into and/or to a blood sample within the microchannel, the first frequency configured to begin separation of red blood cells and plasma in the blood sample, the second frequency configured to complete separation of the red blood cells and plasma, and the third frequency configured to rupture cell walls of the blood cells producing a lysed blood sample.

This application claims benefit under 35 USC § 119(e) of U.S. Provisional Application No. 63/367,347, filed Jun. 30, 2022. The entire contents of the above-referenced patent application are hereby expressly incorporated herein by reference.

### FIELD OF THE DISCLOSURE

The disclosure generally relates to devices, systems, and methods for testing blood samples. More particularly the disclosure relates to an analyzation device configured for separation of red blood cells from plasma in a sample vessel by means of ultrasonic acoustic waves generated in the vessel by a piezo transducer driven at one or more first frequency, or range of frequencies. After separation and testing of the red blood cells and/or plasma, the analyzation device is further configured for lysing red blood cells in the sample vessel by means of ultrasonic acoustic waves, shear forces, pressure, and/or fluid movement, generated in the vessel by a piezo transducer driven at one or more second frequency, or range of frequencies. In some non-limiting embodiments, the ultrasonic acoustic waves are generated by a single piezo transducer. The analyzation device may be used in conjunction with blood sample testing analyzers.

### BACKGROUND

Point-of-care testing refers generally to medical testing at or near the site of patient care, such as in an emergency room. A desired outcome of such tests is often rapid and accurate lab results to determine a next course of action in the patient care. A number of such point-of-care tests involves analysis of a blood sample from the patient. Many of these tests use whole blood, plasma, or serum.

In some tests, the cell walls of red blood cells in the blood sample are ruptured (lysed) to release hemoglobin. Lysis of the red blood cells may be referred to as hemolysis. Typically, hemolysis was done with chemical or mechanical means.

Some devices lyse the red blood cells using ultrasound. Some point-of-care testing devices use spectrophotometric optical absorption measurement for the determination of the Oximetry parameters on a whole blood sample where the red blood cells have been lysed. These devices are fluidic systems that typically position the patient blood sample in a special sample chamber for testing the blood sample. For example, one system described in U.S. Pat. No. 9,097,701 (“Apparatus for Hemolyzing a Blood Sample and for Measuring at Least One Parameter Thereof”, issued Aug. 4, 2015) uses two piezo elements, with two balanced resonant elements, surrounding a sample chamber symmetrically, to lyse the red blood cells using acoustophoretic forces. However, these devices are difficult and expensive to manufacture, including requiring a highly precise symmetry with specially made resonant elements.

Once the red blood cells are lysed, the blood samples may then be tested with a spectrophotometer to analyze the intensity of the predetermined wavelengths of light transmitted through a cartridge optical window. A spectrophotometer is an apparatus for measuring the intensity of light in a part of the spectrum, especially as transmitted or emitted by particular substances. The spectrophotometer measures how much a chemical substance absorbs light by measuring the intensity of light as a beam of light passes through the blood sample, or other solution. Each compound in the sample or solution absorbs or transmits light over a particular range of wavelengths. Absorbance is determined using the Beer-Lambert law. Each compound in the sample or solution absorbs or transmits light over a specific set of wavelengths of interest governed by extinction coefficients.

In such tests, critical-care hematology parameters may be measured that may include hematocrit, free and total hemoglobin, bilirubin, lipids, and oximetry (i.e., E hemoglobin fractions). Doctors and clinicians rely on these measurements to make decisions during patient treatment. These measurements are often performed in a central hematology laboratory on large, complex-to-maintain analyzers. However, obtaining fast, accurate, and precise results in a point-of-care setting is in many ways preferable because it saves time in critical diagnostic situations and avoids specimen transport problems in critical care units. Some blood gas analyzers offer point-of-care capability, but do not present a single solution that provides desired time-to-result, accuracy, precision, and reliability, while being simpler and easier to manufacture than existing devices.

What is needed is an analyzation device to provide improved accuracy and precision of measured parameters of a sample within a desired time-to-result at the point of care of a patient, and that is more easily manufactured and with less cost.

### SUMMARY

Acoustophoretic analyzation devices, methods, and systems are disclosed. The problem of complicated, slow, imprecise, and inaccurate blood sample testing for point-of-care use is addressed through a device configured to separate red blood cells and plasma in a whole blood sample in a sample vessel by means of ultrasonic acoustic waves generated in the sample vessel by a single acoustic transducer, such as a piezo transducer driven at one or more particular first excitation frequency, or range of excitation frequencies. After testing of the separated plasma and/or red blood cells, the analyzation device may be configured to lyse the red blood cells by means of ultrasonic acoustic waves generated in the sample vessel by the single piezo transducer driven at one or more particular second excitation frequency, or range of excitation frequencies. Because the analyzation device may be configured to lyse the red blood cells after testing of the separated plasma and/or red blood cells, the separated plasma and red blood cells are not capable of mixing to reconstitute as whole blood within the analyzation device or within a separate device. The analyzation device may be configured to lyse the red blood cells directly after testing of the separated plasma and/or red blood cells. That is, the same analyzation device may be configured to separate the red blood cells and plasma in the whole blood sample, and thereafter lyse the red blood cells directly after testing of the separated plasma and/or red blood cells.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function. In the drawings:

FIG. 1 is a perspective view of an acoustophoretic analyzation device in accordance with the present disclosure.

FIG. 2 is a top plan view of an acoustophoretic analyzation device in accordance with the present disclosure.

FIG. 3 is bottom plan view of an acoustophoretic analyzation device in accordance with the present disclosure.

FIG. 4 is a first end elevation view of an acoustophoretic analyzation device in accordance with the present disclosure.

FIG. 5 is a second end elevation view of an acoustophoretic analyzation device in accordance with the present disclosure.

FIG. 6 is a first side elevation view of an acoustophoretic analyzation device in accordance with the present disclosure.

FIG. 7 is a cross-sectional view of an exemplary acoustophoretic analyzation device in accordance with the present disclosure.

FIG. 8 is a cross-sectional view of an exemplary acoustophoretic analyzation device in accordance with the present disclosure.

FIG. 9 is a perspective view of components of an exemplary sample vessel in accordance with the present disclosure.

FIG. 10 is a graphical representation of total displacement of an exemplary lysis device in accordance with the present disclosure.

FIG. 11 is a schematic view of an exemplary analyzer in accordance with the present disclosure.

FIG. 12 is a schematic view of components of an exemplary analyzer in accordance with the present disclosure.

FIG. 13 is a diagrammatic view of an exemplary method of blood testing in accordance with one embodiment of the present disclosure.

FIG. 14A is a schematic representation of a whole blood sample inserted in a microchannel in accordance with the present disclosure.

FIG. 14B is a schematic representation of partial separation of red blood cells and plasma in a whole blood sample in the microchannel having been exposed to ultrasonic acoustic waves at a first frequency or first frequency range in accordance with the present disclosure.

FIG. 14C is a schematic representation of substantially complete separation of red blood cells (dotted regions) and plasma (lined regions) in a whole blood sample in the microchannel having been exposed to ultrasonic acoustic waves at a second frequency or second frequency range in accordance with the present disclosure.

FIG. 14D is a schematic representation of a lysed blood sample comprising plasma and red blood cells that have been lysed in the microchannel after having been exposed to ultrasonic acoustic waves at a third frequency or third frequency range in accordance with the present disclosure.

FIG. 15 illustrates one method of calculating an absorption spectrum based on known calculations for absorption for liquid mediums.

FIG. 16 illustrates spectral profile coefficients of the hemoglobin forms.

FIG. 17A is a bottom perspective view of an assembly constructed in accordance with the present disclosure.

FIG. 17B is a top perspective view of the assembly of FIG. 17A.

### DETAILED DESCRIPTION

The following detailed description refers to the accompanying drawings. The same reference numbers in different drawings may identify the same or similar elements.

The mechanisms proposed in this disclosure circumvent the problems described above. The present disclosure describes acoustophoretic analyzation devices, analyzers, and lysis methods, including a acoustophoretic analyzation device configured to first separate red blood cells and plasma in a whole blood sample for testing in a sample vessel by means of ultrasonic acoustic waves generated in the sample vessel by an piezo transducer connected to the sample vessel and driven at one or more first frequency or first range of excitation frequencies followed by lysing the red blood cells in the same sample vessel by means of ultrasonic acoustic waves, shear forces, pressure, cavitation, and/or fluid movement, generated in the sample vessel by the piezo transducer driven at one or more second frequency, or second range of excitation frequencies. In one nonlimiting embodiment, the acoustic transducer, such as a piezo transducer is a single piezo transducer. The present disclosure further describes an analyzer configured to receive and interact with the acoustophoretic analyzation device for testing a sample in the sample vessel, as well as a method of use.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concept. This description should be read to include one or more and the singular also includes the plural unless it is obvious that it is meant otherwise.

Further, use of the term “plurality” is meant to convey “more than one” unless expressly stated to the contrary.

As used herein, qualifiers like “about,” “approximately,” and combinations and variations thereof, are intended to include not only the exact amount or value that they qualify, but also some slight deviations therefrom, which may be due to manufacturing tolerances, measurement error, wear and tear, stresses exerted on various parts, and combinations thereof, for example.

As used herein, the term “substantially” means that the subsequently described parameter, event, or circumstance completely occurs or that the subsequently described parameter, event, or circumstance occurs to a great extent or degree. For example, the term “substantially” means that the subsequently described parameter, event, or circumstance occurs at least 90% of the time, or at least 91%, or at least 92%, or at least 93%, or at least 94%, or at least 95%, or at least 96%, or at least 97%, or at least 98%, or at least 99%, of the time, or means that the dimension or measurement is within at least 90%, or at least 91%, or at least 92%, or at least 93%, or at least 94%, or at least 95%, or at least 96%, or at least 97%, or at least 98%, or at least 99%, of the referenced dimension or measurement.

The use of the term “at least one” or “one or more” will be understood to include one as well as any quantity more than one. In addition, the use of the phrase “at least one of X, V, and Z” will be understood to include X alone, V alone, and Z alone, as well as any combination of X, V, and Z.

The use of ordinal number terminology (i.e., “first”, “second”, “third”, “fourth”, etc.) is solely for the purpose of differentiating between two or more items and, unless explicitly stated otherwise, is not meant to imply any sequence or order or importance to one item over another or any order of addition.

Finally, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As discussed above, typical previous devices for blood sample testing for point-of-care use are complicated, slow, imprecise, and inaccurate. The present disclosure addresses these deficiencies with devices, systems, and methodology for separating plasma from red blood cells in whole blood in a sample vessel by means of ultrasonic acoustic waves generated by an piezo transducer connected to the sample vessel driven at a first particular excitation frequency having power sufficient to begin separation of the red blood cells from the plasma, followed by a second excitation frequency at a power level sufficient to substantially complete separation of the red blood cells from the plasma, running at least one first test, then lysing the red blood cells in the sample vessel by means of the ultrasonic acoustic waves at a third excitation frequency at a power level sufficient to lyse the red blood cells thereby producing a lysed blood sample on which one or more second tests may be run.

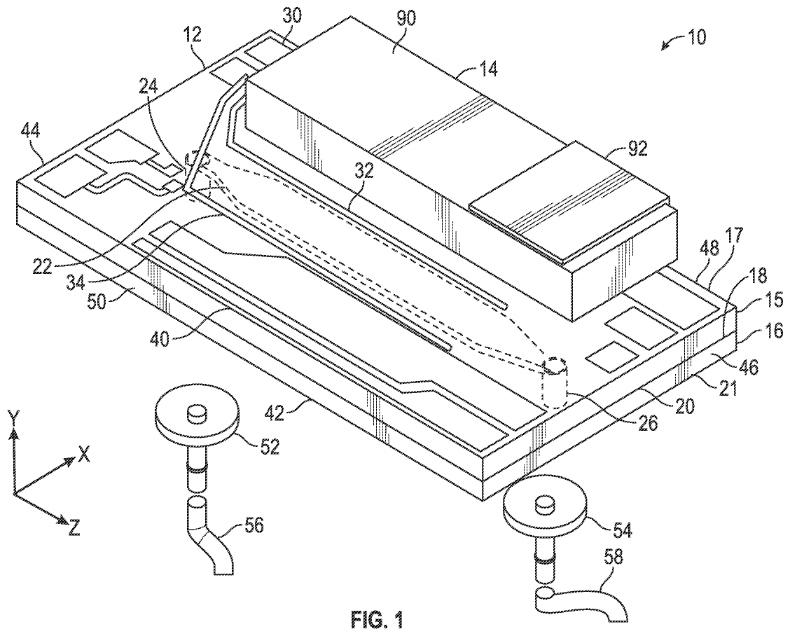

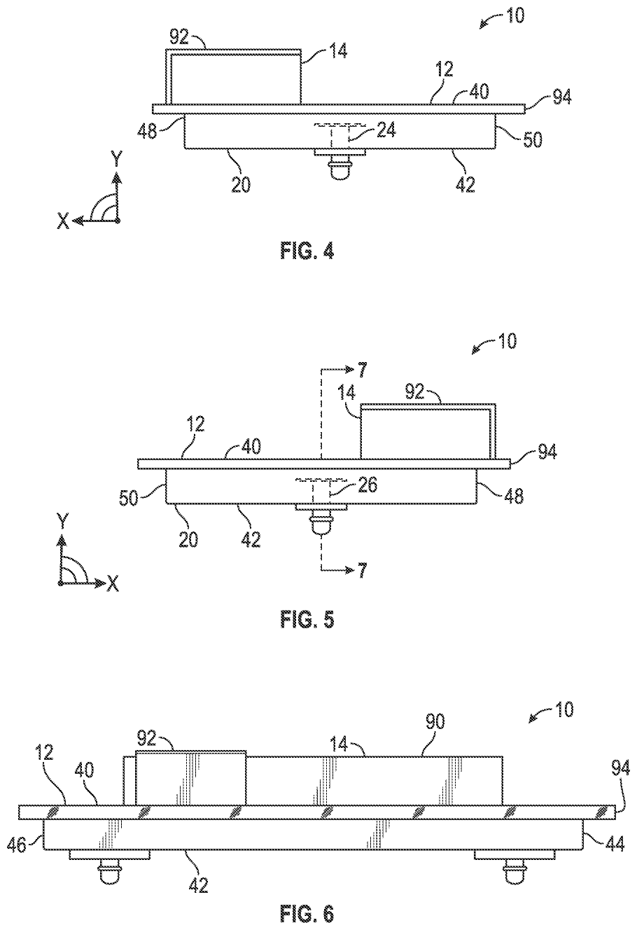

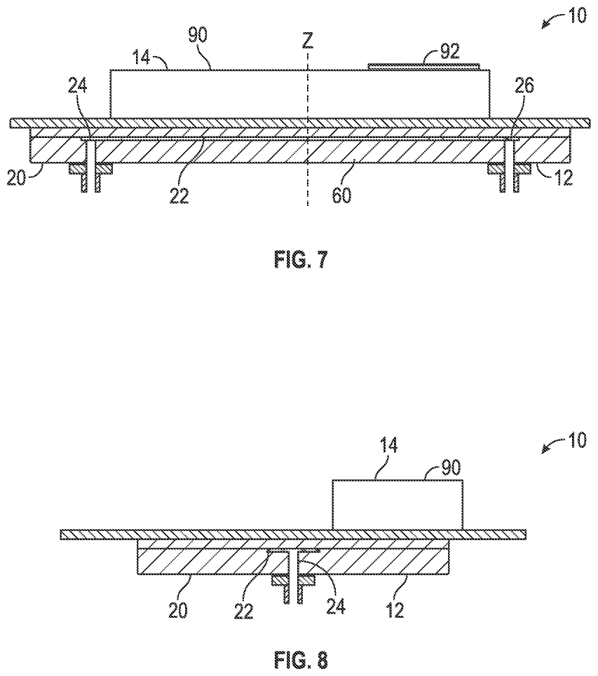

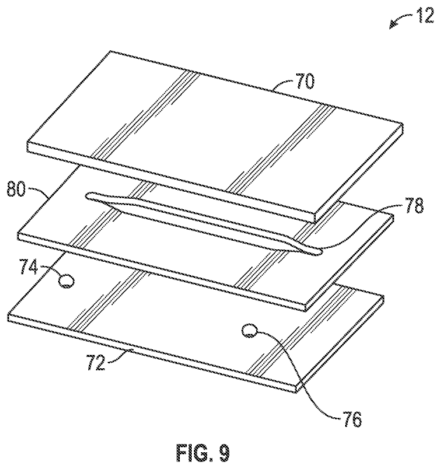

Referring now to the drawings, and in particular to FIGS. 1-8, an acoustophoretic device 10 is shown. In general, the acoustophoretic device 10 comprises a sample vessel 12 and an acoustic transducer 14 which may be a piezo transducer 14. The acoustic transducer 14 will be referred to herein as the piezo transducer 14. The piezo transducer 14 is bonded to the sample vessel 12. In one embodiment, the acoustophoretic device 10 is a monolithic structure, such as that formed by the sample vessel 12 and the piezo transducer 14 bonded together using a suitable bonding material, such as epoxy. The sample vessel 12 is preferably permanently bonded to the piezo transducer 14 in a non-clamping manner. This non clamping manner is done in order to minimize vibrational losses between the sample vessel 12 and the piezo transducer 14.

The sample vessel 12 is provided with a first substrate 15 bonded to a second substrate 16 using a suitable bonding material, such as epoxy. The first substrate 15 having an upper surface 17 and a lower surface 18. The second substrate 16 having an upper surface 20, a lower surface 21, a microchannel 22 within the confines of the upper surface 20, a first port 24 extending from the lower surface 21 to the microchannel 22 and in fluid communication with the microchannel 22, and a second port 26 extending through the lower surface 21 to the microchannel 22 and in fluid communication with the microchannel 22. In one embodiment, the upper surface 17 of the first substrate 15 may have a mounting area for the piezo transducer 14.

In one embodiment, the sample vessel 12 has a top 40, a bottom 42, a first end 44, a second end 46, a first side 48, and a second side 50, wherein the first side 48 and the second side 50 extend between the first end 44 and the second end 46 and between the top 40 and the bottom 42. In one embodiment, the top 40 and the bottom 42 are planar. In one embodiment, the first side 48 and the second side 50 are planar. In one embodiment, the first end 44 and the second end 46 are planar. In one embodiment, the top 40, the bottom 42, the first end 44, the second end 46, the first side 48, and the second side 50 cooperate to form a three-dimensional rectangular cuboid. In some embodiments, the piezo transducer 14 matingly engages an outer surface of the sample vessel 212. For example, the piezo transducer 14 and the outer surface may have planar surfaces configured to be positioned together.

The sample vessel 12 may be partially, substantially, or completely transparent. In one embodiment, the sample vessel 12 is transparent at least above and below the microchannel 22, such that a light beam may pass through the sample vessel 12 through the microchannel 22, interact with any substance within the microchannel 22, and pass out of the sample vessel 12.

The sample vessel 12 may be constructed of glass. In one embodiment, the sample vessel 12 may be constructed of a material (glass or non-glass) having a Young's modulus within a range from about 50 Gpa to about 90 Gpa. The material property known as Young's modulus, or the modulus of elasticity, is a measure of the ability of the material to withstand changes in length when under lengthwise tension or compression. Young's modulus is equal to the longitudinal stress divided by the strain. In one embodiment, the sample vessel 12 may be constructed of plastic with a rigidity and/or Young's modulus similar to that of glass. In one embodiment, the sample vessel 12 may be constructed from alkali borosilicate glass. One example of alkali borosilicate glass is made by Schott Advanced Optics, located at 400 York Avenue, Duryea, PA 18642, and marketed under the name “D 263 T ECO Thin Glass.”

In one embodiment, the sample vessel 12 may have one or more conductive structure 30, that may be metal sputtered or otherwise bonded to the upper surface 17 of the first substrate 15. The one or more conductive structure 30 provide electrical pathways that may connect elements such as the piezo transducer 14. The one or more conductive structure 30 may include a first electrode 32 and a second electrode 34 positioned on either side and running substantially parallel to the microchannel 22. The first electrode 32 and the second electrode 34 may extend within plus or minus 5 degrees of parallel (preferably extending parallel). In the embodiments shown, the first electrode 32 and the second electrode 34 are both in a linear configuration and adjacent to but not covering the microchannel 22 so as to not block a beam of light generated by a spectrophotometer as discussed herein. In some embodiments, the first electrode 32 and the second electrode 34 may be parallel or non-parallel so long as the first electrode 32 and the second electrode 34 do not block a light beam 116, and permit capacitance readings from the first electrode 32 and the second electrode 34 to be correlated to expected channel contents within the microchannel 22 during calibration. In some embodiments, the first electrode 32 and/or the second electrode 34 can have a serpentine configuration with one or more portion(s) crossing above the microchannel 22 but outside of an expected path of the light beam 116 through the sample vessel 12.

The sample vessel 12 has a length from the first end 44 to the second end 46, a width from the first side 48 to the second side 50, a thickness between the top 40 and the bottom 42, and an aspect ratio defining the proportional relationship between the length and the width. The sample vessel 12 has a longitudinal axis along the length and a latitudinal axis along the width.

In one embodiment, the aspect ratio of the sample vessel 12 is in a range from approximately 0.5 to approximately 3.0. In one embodiment, the aspect ratio of the sample vessel 12 is in a range from approximately 1.4 to approximately 1.9. In one embodiment, the length may be approximately twenty-two millimeters and the width may be approximately twelve millimeters. In one embodiment, the length may be approximately seventeen millimeters and the width may be approximately twelve millimeters. In one embodiment, the length may be approximately seventeen millimeters and the width may be approximately six millimeters. In one embodiment, the length may be approximately twelve millimeters and the width may be approximately six millimeters.

The microchannel 22 may be configured to receive a fluidic sample (including, but not limited to, a blood sample, a “blank” sample, and/or a washing solution sample) through the first port 24 and/or the second port 26. An inlet 52 may be connected to the first port 24 and an outlet 54 may be connected to the second port 26 to allow fluidic connection of a first tube 56 and a second tube 58, respectively, to the microchannel 22. The microchannel 22 has a length, a width, and a height. Typically, the length of the microchannel 22 is oriented along the longitudinal axis of the sample vessel 12 and the width of the microchannel 22 is oriented along the latitudinal axis of the sample vessel 12. However, it will be understood that the microchannel 22 may be oriented at an angle from or offset from the longitudinal axis and/or the latitudinal axis of the sample vessel 12.

The microchannel 22 has an aspect ratio defining the proportional relationship between the width and the height of the microchannel 22. In one embodiment, the width to height aspect ratio of the microchannel 22 is in a range from approximately 0.04 to approximately 0.175. In one embodiment, the width to height aspect ratio of the microchannel 22 is in a range from approximately 0.04 to approximately 0.125. In one embodiment, the width to height aspect ratio of the microchannel 22 is approximately 0.05.

In one embodiment, the width of the microchannel 22 is about two millimeters. In one embodiment, the width of the microchannel 222 is about 2.5 millimeters. In one embodiment, the width of the microchannel 22 is greater than an illumination width of a light yield area of an absorbance spectrophotometer 102. An illumination width may be defined as the width of a cross-section of the light yield along an optical pathway from the absorbance spectrophotometer 102 where the optical pathway intersects the microchannel 22. For example, when the illumination diameter is between 1 millimeter and 1.5 millimeter, then the width of the microchannel 22 may be at least approximately 1.6 millimeters. The width of the microchannel 22 may be determined to allow for adequate mechanical alignment between the microchannel 22 and optical pathway. For example, for an illumination width between 1 millimeter and 1.5 millimeter, the width of the microchannel 22 may be approximately two millimeters.

In one embodiment, the length of the microchannel 22 may be between approximately ten millimeters and approximately twelve millimeters. In one embodiment, the length of the microchannel 22 may be at least approximately four millimeters. In one embodiment, the length of the microchannel 22 may be between approximately four millimeters and approximately twenty millimeters.

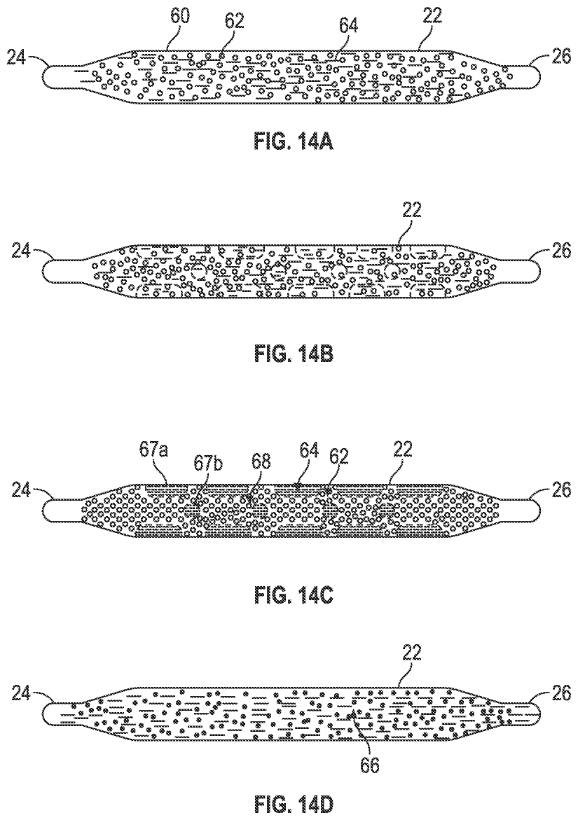

In one embodiment, the length of the microchannel 22 may be based at least in part on a predetermined desired number of acoustic nodes to be created in the microchannel 22. For example, for a microchannel 22 having a width of approximately two millimeters and where a whole blood wave propagation speed is approximately 1500 m/s, a calculated single acoustic node is at 350 kHz. The acoustic nodes may be distributed in the microchannel 22 evenly spaced along the length of the microchannel 22 (for example, 2×2 mm=4 mm), where high pressure creates a uniform distribution of lysed blood. For example, if the predetermined desired number of acoustic nodes is six nodes on each side wall of the microchannel 22, in the region where the sidewalls run approximately parallel (see FIG. 14C depicting five anti-node region(s) 67a and the six nodes 68 which include two end nodes and four nodes between the five anti-node regions 67a), then the length of the microchannel 22 may be set at approximately seventeen millimeters including tapered inlet and outlet regions.

The height of the microchannel 22 can vary, as discussed below. The height of the microchannel 22 may be based on the amount of absorption in lysed blood of the light yield from the absorbance spectrophotometer 102 and the desired precision of the absorption. For example, the desired absorption may be at approximately 1 Optical Density (OD).

In one embodiment, the height of the microchannel 22 is about 100 micrometers. In one embodiment, the height of the microchannel 22 is about 150 micrometers. In one embodiment, the height of the microchannel 22 is about 250 micrometers. In one embodiment, the height of the microchannel 22 is about 300 micrometers. In one embodiment, the height of the microchannel 22 is between approximately 80 micrometers and approximately 300 micrometers. In one embodiment, the height of the microchannel 22 is between approximately 80 micrometers and approximately 150 micrometers.

The first port 24 and the second port 26 are fluidly connected to the microchannel 22 and extend from the microchannel 22 through the lower surface 21 of the second substrate 16. In one embodiment, the first port 24 is fluidly connected to the microchannel 22 and may extend from the microchannel 22 to the top 40, the bottom 42, the first end 44, the second end 46, the first side 48, and/or the second side 50 of the sample vessel 12. In one embodiment, the second port 26 is fluidly connected to the microchannel 22 and may extend from the microchannel 22 to the top 40, the bottom 42, the first end 44, the second end 46, the first side 48, and/or the second side 50 of the sample vessel 12. The first port 24 and the second port 26 may extend to the same or to different ones of the top 40, the bottom 42, the first end 44, the second end 46, the first side 48, and/or the second side 50.

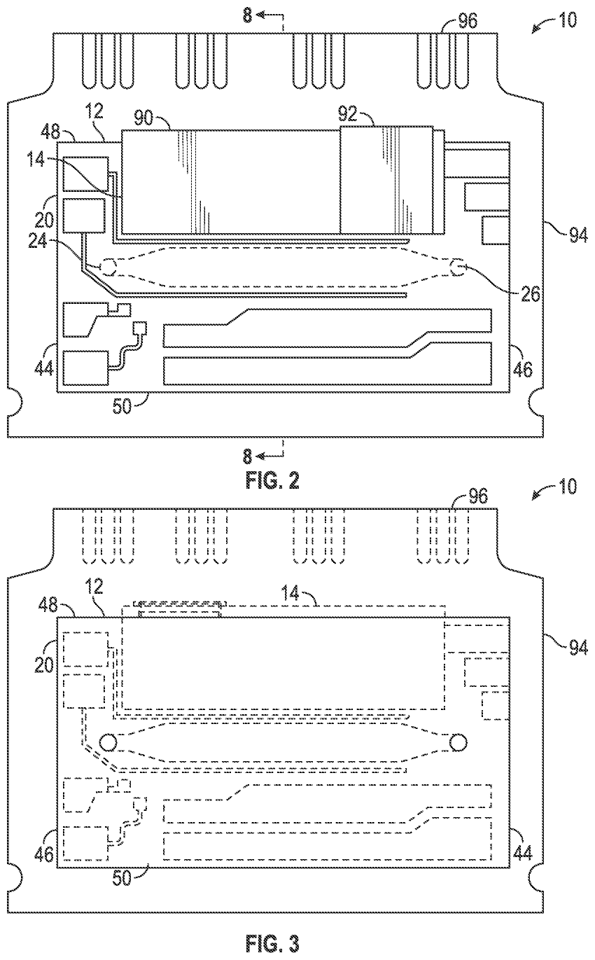

In one embodiment, the first port 24 and the second port 26 each have a diameter of between approximately 0.5 millimeter (500 micrometers) and approximately 1.5 millimeter (1500 micrometers). In one embodiment, the first port 24 and the second port 26 each have a diameter of approximately 0.8 millimeter (800 micrometers). The microchannel 22 tapers toward the first port 24 and towards the second port 26, such as shown in FIG. 2. The taper assists in providing the fluid to and/or from the first port 24. The cross-sectional width (e.g., diameter) of the first port 24 and the second port 26 are smaller than the width of the microchannel 22. For example, a cross-sectional width of the first port 24 and the second port 26 can be from 50% to 100% of the width of the microchannel 22.

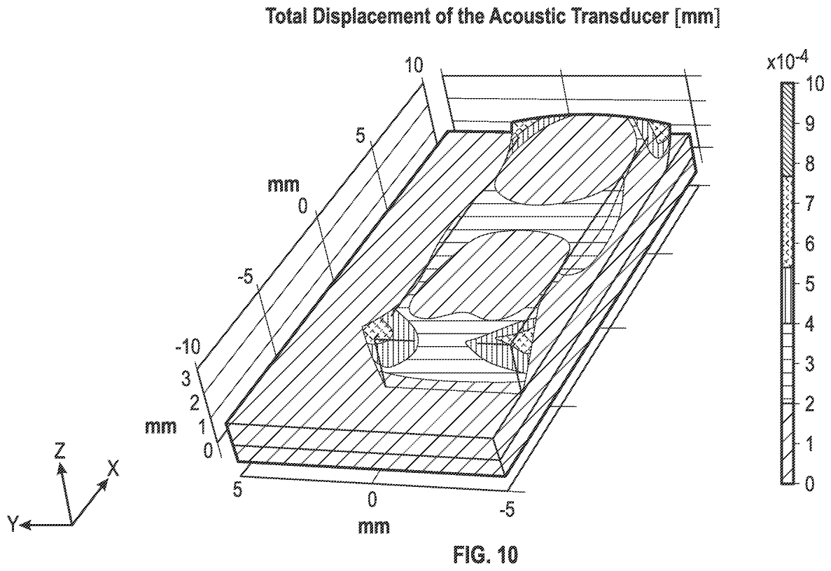

The sample vessel 12 may be a monolithic fabrication, either in that the sample vessel 12 is formed from a single piece of material or in that the sample vessel 12 is formed from a plurality of pieces that are interconnected to form a unified whole. As discussed, with respect to FIG. 1, the sample vessel 212 may be formed from two substrates that are bonded together. Alternatively, the sample vessel 12 may be formed from three substrates that are bonded together, as shown in FIG. 9.

The second substrate 16 may be layered with the first substrate 15 so as to form a monolithic structure. In one embodiment, the first substrate 15 and the second substrate 16 may be annealed to one another. In one embodiment, the first substrate 15 and the second substrate 16 may be thermal-plasma bonded to one another. In one embodiment, the first substrate 15 and the second substrate 16 have the same length to width aspect ratio as the sample vessel 12.

The microchannel 22 may be positioned in the first substrate 15, the second substrate 16, and/or be formed partially in the first substrate 15 and partially in the second substrate 16. In one embodiment, the microchannel 22, the first port 24, and the second port 26 are positioned in the first substrate 15. In one embodiment, the microchannel 22 is etched into the first substrate 15 and/or the second substrate 16. In one embodiment, the microchannel 22 is positioned in the first substrate 15 and one or both of the first port 24 and the second port 26 is positioned in the second substrate 16. One or both of the first port 24 and the second port 26 may be positioned in (and/or extend through) the first substrate 15 and/or the second substrate 16.

As illustrated in FIG. 9, in one embodiment, the sample vessel 12 may comprise a first substrate 70, a second substrate 72, and a third substrate 80 between the first substrate 70 and the second substrate 72. The first substrate 70, the second substrate 72, and the third substrate 80 may be layered so as to form a monolithic structure. In one embodiment, the first substrate 70, the second substrate 72, and the third substrate 80 may be thermal-plasma bonded to one another. In one embodiment, the first substrate 70, the second substrate 72, and the third substrate 80 may be annealed to one another. One or both of a first port 74 and a second port 76 may be positioned in the second substrate 72. In one embodiment, A microchannel 78 is a slot positioned through the third substrate 80. In one embodiment, the third substrate 80 may have a same thickness as a height of the microchannel 78. In one embodiment, the third substrate 80 may be 100 micrometers thick. In one embodiment (not shown), the microchannel 78 may be positioned in the second substrate 72.

Returning to FIG. 1, the piezo transducer 14 is mounted to the sample vessel 12 (such as to a mounting area of the top 40) to form the monolithic structure of the acoustophoretic device 10. The piezo transducer 14 may have a mounting surface that mounts to the mounting area of the top 40. In one embodiment, the piezo transducer 14 is mounted at least partially to the top 40 of the sample vessel 12; however, it will be understood that the piezo transducer 14 may be mounted to the top 40, the bottom 42, the first end 44, the second end 46, the first side 48, and/or the second side 50. The piezo transducer 14 is positioned in relation to the microchannel 22 such that it does not block light from moving through the microchannel 22 from the top 40 or the bottom 42 of the sample vessel 12. The piezo transducer 14 may be offset from the microchannel 22 such that the piezo transducer 14 allows light to enter the microchannel 22 from outside of the sample vessel 12. In one embodiment, the piezo transducer 14 has a length and has a longitudinal axis along the length that is orientated substantially parallel (e.g., within 5 degrees of parallel) to the longitudinal axis of the sample vessel 12. In one embodiment, the piezo transducer 14 has a width that is smaller than the length of the piezo transducer 14.

The piezo transducer 14 may be positioned on the opposite side from one or both of the first port 24 and the second port 26 or on the same side as one or more of the first port 24 and the second port 26 on the sample vessel 12

The piezo transducer 14 may be bonded to the sample vessel 12. The bond may be thin relative to a thickness of the piezo transducer 14 and the sample vessel 12. The piezo transducer 14 may be bonded to the sample vessel 12 with an adhesive. The adhesive may be configured to allow acoustic wave propagation with low attenuation of acoustic waves. In one embodiment, a liquid adhesive may be applied to the piezo transducer 14 and then the piezo transducer 14 may be attached via the liquid adhesive to the sample vessel 12. For example, a liquid adhesive having temperature stability up to 350° C., excellent adhesive force on glass, and high hardness (rigidity) may be applied. In one example, the liquid adhesive may be an epoxy glue, such as EPO-TEK 353ND (made by Epoxy Technology, Inc., located at 14 Fortune Drive, Billerica, MA), which allows for ultrasound propagation and which has a shore D hardness of about 85. In one example, approximately 5 μl of liquid adhesive may be applied. The piezo transducer 14 may be clamped to the sample vessel 12 and the adhesive cured at approximately 150° C. In one implementation, after curing, the thickness of the adhesive may be approximately 100 μm. In one implementation, after curing, the thickness of the adhesive may be approximately 10 μm.

The piezo transducer 14 may be configured to convert an applied alternating electrical field into another form of energy, such as acoustic pressure waves having one or more frequency and/or a range of frequencies. The piezo transducer 14 may be configured to oscillate when an alternating electrical field is applied to the piezo transducer 14, thereby creating the acoustic pressure waves that are introduced into the sample vessel 12, which may create one or more standing acoustic node within the blood sample in the sample vessel 12. As shown in FIG. 1, the piezo transducer 14 may comprise a first terminal 90 and a second terminal 92 configured to connect with an alternating voltage source. In one embodiment, the first terminal 90 and the second terminal 92 may be electrically connected to the conductive structures 30 to deliver the alternating voltage. In one embodiment, the piezo transducer 14 may be a piezoelectric ultrasonic transducer.

The piezo transducer 14 may be configured to generate mechanical activity, producing acoustic waves with certain frequencies, by expanding and contracting when an alternating electrical field is applied. FIG. 10 shows a graphical representation of one example of the total displacement of the piezo transducer 14 in one exemplary operation of the piezo transducer 14.

In one embodiment, the piezo transducer 14 may be configured to produce ultrasonic waves having a first frequency that may be in a range of between 950 kHz and 1100 kHz that begins to cause separation of plasma and other constituents of the blood sample such as red blood cells for a first predetermined time period. The beginning of separation of plasma and other constituents of the blood sample suspending within the plasma may be referred to herein as pre-enrichment, and the first frequency may be referred to herein as the pre-enrichment frequency. The first frequency may be produced with a first electrical voltage that may be between 50 volts and 100 volts. The first predetermined time period may be between 5 seconds and 15 seconds. In one embodiment, the first frequency may be in a range of between 600 kHz and 630 kHz.

The piezo transducer 14 may further be configured to emit ultrasonic waves at a second frequency and/or range of frequencies and at a second electrical voltage for a second predetermined period of time causing red blood cells suspended within the plasma in the blood sample to completely separate. The complete separation of plasma and other constituents of the blood sample suspending within the plasma may be referred to herein as enrichment, and the second frequency may be referred to herein as the enrichment frequency. In one embodiment, the second frequency and/or range of frequencies may be different (e.g., lower) than the first frequency or range, the second electrical charge may be different (e.g., higher) than the first electrical charge, and the second predetermined time period may be the same or different (e.g., greater) than first predetermined time period. By way of example, the second frequency and/or range of frequencies may be between 320 kHz and 500 kHz and the second electrical charge may be between 70 volts and 100 volts. The second predetermined time period may be between 10 and 25 seconds.

In one embodiment, the second frequency may cause one or more acoustic standing wave which is different than the acoustic standing wave caused by the first frequency. An acoustic standing wave, also known as a stationary wave, is a wave that oscillates in time, but that has a peak amplitude profile that does not move in space. The acoustic standing wave in the microchannel 22 may form node regions having approximately zero force and approximately no particle movement and anti-node regions having a highest force and the most particle movement relative to the rest of the microchannel 22. As the red blood cells and the plasma in the blood sample separate when the second frequency is applied, the red blood cells tend to move to the node regions in the microchannel 22 and the plasma tends to move to the anti-node regions. This separation into known regions at specific and repeatable locations within the microchannel 22 allows plasma and/or red blood cell analyte measurement as will be described further below.

It should be noted that the first frequency and the second frequency may together be referred to herein as the separation frequency, that is, the frequencies of the ultrasonic or acoustic waves imparted into the sample vessel sufficient to separate red blood cells and plasma of the whole blood sample without rupturing the red blood cells.

The piezo transducer 14 may further be configured to emit ultrasonic waves at a third frequency and/or range of frequencies and at a third electrical voltage for a third predetermined period of time subsequent to the first predetermined period of time and the second predetermined period of time causing red blood cells in the blood sample to lyse. The third frequency and/or range of frequencies may be referred to herein as the lysis frequency. The piezo transducer 14, when driven with the third electrical charge generates acoustic standing waves within the microchannel 22 that are configured to rupture the cell walls of a cell in the blood sample, and configured to release hemoglobin from within the cell that mixes with the plasma. In one embodiment, the third frequency and/or range of frequencies may be different (e.g., lower) than the first and/or second frequencies or ranges, the third electrical voltage may be different (e.g., higher) than the first electrical voltage and the same or different (e.g., higher or lower) than second electrical charge, and the third predetermined time period may be the same or different (e.g., less) than first or second predetermined time periods. By way of example, the third frequency and/or range of frequencies may be between 330 kHz and 370 kHz and the third electrical charge may be between 100 volts and 200 volts. The third predetermined time period may be less than 20 seconds, such as between 0, 1 or 2 and 10 seconds.

In one non-limiting exemplary embodiment, to cause pre-enrichment, the first frequency may be from 1092 kHz to 1097 kHz which may be produced with a first electrical voltage that may be 70 volts and during the first predetermined time period of 6 seconds. To cause enrichment, the second frequency and/or range of frequencies may be from 376 kHz to 377 kHz which may be produced with a second electrical voltage that may be 80 volts and during the second predetermined time period of 12 seconds. To cause lysis of the red blood cells, the third frequency and/or range of frequencies may be from 349 kHz to 351 kHz which may be produced with a third electrical voltage that may be 120 volts.

In another non-limiting exemplary embodiment, to cause pre-enrichment, the first frequency may be from 1092 kHz to 1097 kHz which may be produced with a first electrical voltage that may be 90 volts and during the first predetermined time period of 6 seconds. To cause enrichment, the second frequency and/or range of frequencies may be from 376 kHz to 377 kHz which may be produced with a second electrical voltage that may be 100 volts and during the second predetermined time period of 12 seconds. To cause lysis of the red blood cells, the third frequency and/or range of frequencies may be from 349 kHz to 351 kHz which may be produced with a third electrical voltage that may be 120 volts.

In a further non-limiting exemplary embodiment, to cause pre-enrichment, the first frequency may be from 600 kHz to 630 kHz, and more particularly from 612 kHz to 613 kHz which may be produced with a first electrical charge that may be 5 to 12 volts (e.g., 6 or 10 volts) and during the first predetermined time period of 8 seconds. To cause enrichment, the second frequency and/or range of frequencies may be from 470 kHz to 500 kHz, and more particularly from 486 kHz to 487 kHz which may be produced with a second electrical charge that may be 13 to 20 volts (e.g., 13 or 18 volts) and during the second predetermined time period of 15 seconds. To cause lysis of the red blood cells, the third frequency and/or range of frequencies may be from 343 kHz to 348 kHz which may be produced with a third electrical charge that may be 13 volts.

The third frequency may be a resonant frequency that causes resonances in the blood sample that is introduced into the microchannel 22 of the sample vessel 12 such that walls of red blood cells in the blood sample are ruptured. In one embodiment, the piezo transducer 14 has a first resonant frequency and the monolithic structure of the acoustophoretic device 10 has a second resonant frequency spaced spectrally from the first resonant frequency, the second resonant frequency being a frequency of acoustic waves that are generated by the piezo transducer 14 and introduced into the sample vessel 12 thereby causing cavitation in the blood sample generating bubbles that collapse in regions with higher pressure and create shock waves which then lead to rupturing the walls of the red blood cells releasing hemoglobin from within the red blood cells into the plasma of the blood sample.

In one example, at the main resonance of the acoustophoretic device 10 (that is, the sample vessel 12 bonded to the piezo transducer 14), for example, when the sample vessel 12 is made of glass, the microchannel 22 has a width of approximately two millimeters with an aspect ratio of 0.05 to 0.125, and the sample vessel 12 has a width of approximately twelve millimeters with an aspect ratio of 1.4 to 1.9., the piezo transducer 14 may be configured to produce ultrasonic sound waves in the range of 330 kHz to 370 kHz with peak pressure within the microchannel 22 of five MPa, and peak velocity up to eight m/s.

However, ultrasonic acoustic waves inside the microchannel 22 and the ultrasonic piezo transducer 14 may produce undesired heat, including undesired heat in the blood sample in the microchannel 22. To avoid overheating of the blood sample, the piezo transducer 14 may be operated to produce a resonant frequency for a predetermined period of time. For example, the piezo transducer 14 may be operated to generate acoustic waves having the second resonant frequency for between approximately one second and approximately two seconds. In one embodiment, the piezo transducer 14 may be operated to generate acoustic waves having the second resonant frequency for less than approximately one and a half seconds. In one example, the acoustophoretic device 10 may be configured to operate the piezo transducer 14 for equal to or less than 1.5 seconds to result in 99.99% red blood cell lysis. In one example, the acoustophoretic device 10 may be configured to operate the piezo transducer 14 for approximately ten seconds or less.

In one embodiment, the ultrasonic sound waves inside the microchannel 22 disrupt the blood cells and cell walls into fine particles which produce less light scattering during optical measurement of the blood sample than larger particles.

In one embodiment, the piezo transducer 14 may be configured to produce ultrasonic acoustic waves in a range of frequencies and the second resonant frequency may be within the range of frequencies.

In one embodiment, the piezo transducer 14 may be configured to produce ultrasonic sound waves in a range of frequencies that is within a range of frequencies between 300 kHz-1.5 MHz.

The resonant frequency, and/or the frequency range, may be determined based on one or more factors including the size, shape, and material of the sample vessel 12; the size and shape of the microchannel of the sample vessel 12; the amount of fluid in the fluidic sample; and/or the size, shape, and material of the piezo transducer 14; and/or the size, shape, and material of the adhesive layer between sample vessel and piezo transducer.

For example, when the sample vessel 12 is made of glass, the microchannel 22 has an aspect ratio of approximately 0.05 to approximately 0.125, and the sample vessel 12 has an aspect ratio of approximately 1.4 to approximately 1.9., the piezo transducer 14 may be configured to produce ultrasonic acoustic waves in the range of approximately 330 kHz to approximately 370 kHz.

The width of the microchannel 22 may be determined based at least on acoustic wave propagation speed inside the blood sample (for example, approximately 1500 m/s) and using the predetermined desired number of acoustic nodes as one node in the middle of the microchannel 22, such that the frequency is approximately 330 kHz to approximately 370 kHz. The following formula may be used to determine, at least in part, a first acoustic node inside the microchannel 22 (with an exemplary 2000 μm width and 100 μm depth), without considering any minor reflection or other mirroring:

$$f = v / λ$$

where f is the frequency, v the wave speed in fluid and λ the wavelength (where the wavelength is two times the width of the microchannel 22, for a half-wavelength resonance mode with a single node over the width of the channel).

Because the resonant frequency of the sample vessel 12 may be difficult to calculate precisely due to manufacturing and/or material variances, in one embodiment, the piezo transducer 14 may be configured to sweep the third frequency within frequency ranges having a plurality of frequencies, starting at a first sweep frequency and proceeding through one or more second sweep frequencies to a third sweep frequency of the plurality of frequencies. In one embodiment, the piezo transducer 14 may be configured to sweep the third frequency range in steps, such as steps of one kHz of frequency. In one embodiment, sweeping the third frequency range from the first sweep frequency to the third sweep frequency ensures that the resonant frequency for the acoustophoretic device 10 plus the blood sample is reached, even in light of variances in the geometry and materials of the acoustophoretic device 10.

In one embodiment, the piezo transducer 14 may be configured to sweep the third frequency range between approximately 330 kHz and approximately 370 kHz, such as, in approximately one kHz steps. The piezo transducer 14 may be configured to sweep the third frequency range starting approximately 330 kHz and going to approximately 370 kHz and/or the piezo transducer 14 may be configured to sweep the third frequency range starting approximately 370 kHz and going to approximately 330 kHz, for example.

In one embodiment, the piezo transducer 14 may be configured to sweep the third frequency range over a time period greater than zero seconds, and less than five seconds, less than four seconds, less than three seconds, less than two seconds, and/or less than one second. In one embodiment, the piezo transducer 14 may be configured to sweep the third frequency range in a time period between approximately one and approximately two seconds.

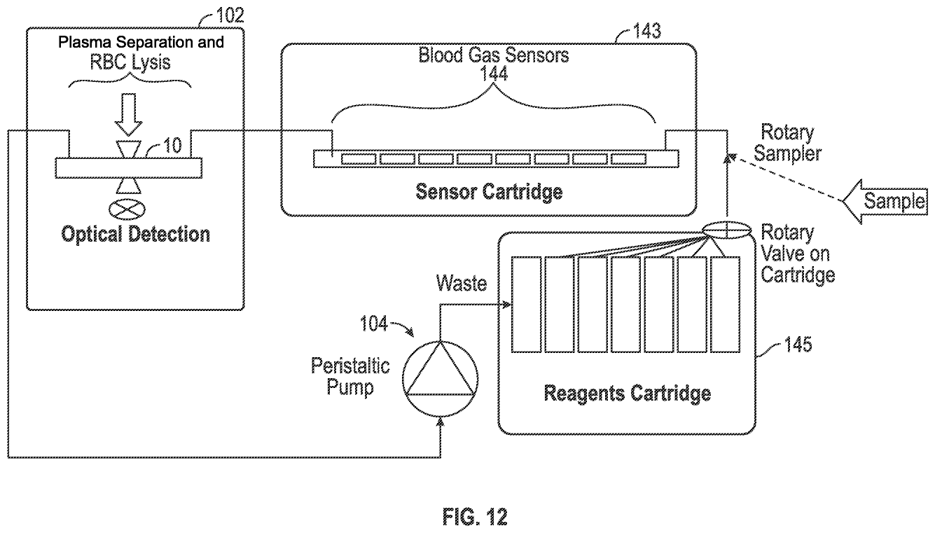

In one embodiment, additionally or alternatively, the acoustophoretic device 10 may lyse the blood cells in the blood sample by inducing shear and vibrating modes in the microchannel 22 of the sample vessel 12. Displacement of the rigid bonded ultrasonic piezo transducer 14, which may be primarily transverse displacement, causes vibration and movement of the sample vessel 12 bonded to the piezo transducer 14. When activated, the ultrasonic piezo transducer 14 changes shape, contracting and elongating (transverse displacement) as shown in FIG. 12. The movement of the ultrasonic piezo transducer 14, is translated to the sample vessel 12, changing the geometry and/or volume of the microchannel 22, which induces shear and vibrating in the microchannel 22 of the sample vessel 12. FIG. 12 shows a graphical representation of one example of the total displacement of the piezo transducer 14 in one exemplary operation of the piezo transducer 14.

The displacement of the piezo transducer 14 may result in vibrating of, and shear forces within, the sample vessel 12, which subsequently may cause and/or contribute to lysis of the blood sample in the microchannel 22 of the sample vessel 12 due to a combination of high pressure, shear forces, and/or fluid movement inside the microchannel 22. Therefore, in some implementations, lysis of the blood sample in the microchannel 22 may be caused by a combination of acoustic standing waves, pressure, cavitation, shear forces, and/or fluid movement within the blood sample.

Shear stress may be developed at the bond between the piezoelectric piezo transducer 14 and the sample vessel 12 when the piezo transducer 14 is activated. The shear stress may result in high pressures inside of the microchannel 22. For example, in one embodiment, a preferred high pressure may be approximately 5 MPa. In one embodiment, the pressure may be in a range of approximately 3 MPa to approximately 7 MPA. The level of pressure may be controlled by the level of contraction/elongation of the piezo transducer 14, which may depend on the electric field strength of the piezo transducer 14.

The combination of acoustic standing waves inside the microchannel 22 along with shear and/or vibrating of the sample vessel 12 causes significant cavitation and dependent shock waves in the whole blood sample in the microchannel 22, which causes the cell walls of the red blood cells to rupture releasing hemoglobin from the red blood cells.

In one embodiment illustrated in FIGS. 2-8, the acoustophoretic device 10 may be bonded or otherwise connected to a printed circuit board (PCB) 94. The acoustophoretic device 10 may be constructed in a manner identical to the acoustophoresis device 204 described in U.S. Ser. No. 63/366,552, the entire content of which is hereby incorporated herein by reference. The PCB 94 may be provided with electrically conducting structures (not shown) on an outer surface or embedded inside the PCB 94 as is known in the art. The electrically conducting structures of the PCB 94 may electrically connect the conductive structures 30 of the acoustophoretic device 10 to one or more electrical connector 96 (only one of which is numbered). The one or more electrical connector 96 may be configured to connect the acoustophoretic device 10 to an analyzer such as analyzer 100 that will be described further herein. The PCB 94 may be constructed in a manner identical to the support substrate 202 described in U.S. Ser. No. 63/366,552. Additionally, the acoustophoresis device 10 may be attached to the PCB 94 by soldering mounting pads on the sample vessel 12 to mounting pads on the PCB 94 in a manner described in U.S. Ser. No. 63/366,552.

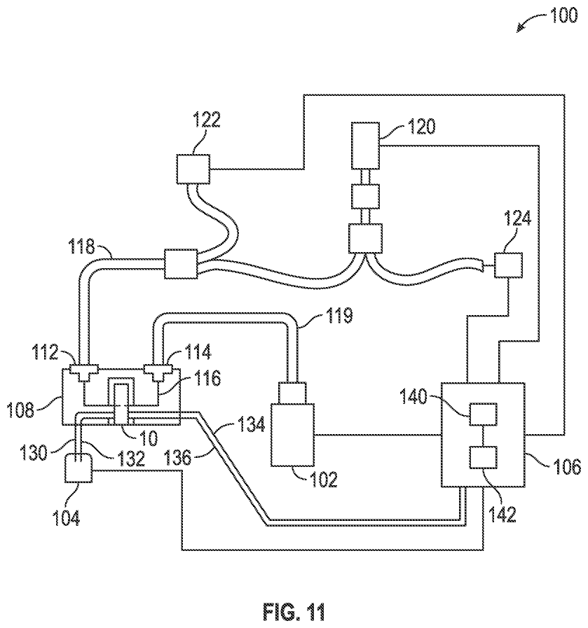

Referring now to FIGS. 11 and 12, in some embodiments, the acoustophoretic device 10 may be a component of an analyzer 100. The analyzer 100 may comprise the acoustophoretic device 10, an absorbance spectrophotometer 102, a fluidic distribution system 104 (for example, including a peristatic pump), and/or a controller 106. In one embodiment, the acoustophoretic device 10 is removable and/or exchangeable from the other components of the analyzer 100. In one embodiment, the analyzer 100 may further comprise a mount 108 configured to receive the PCB 94 supporting the other components of the acoustophoretic device 10. In one embodiment, the acoustophoretic device 10 may be held by the mount 108 such that the acoustophoretic device 10 is able to vibrate and/or move within a range of vibration and/or movement.

In one embodiment, the analyzer 100 may further comprise one or more processors 140 coupled to one or more non-transitory computer readable medium 142 storing instructions that are provided to the one or more processors 140 and when executed by the one or more processors 140 cause the one or more processors 140 to perform functions described hereinafter. In one embodiment, the one or more processors 140 and the one or more non-transitory computer readable medium 142 may be part of the controller 106. However, it will be understood that one or more of the processors 140 and/or the non-transitory computer readable medium 142 may be located external to the controller 106 and/or external to the other components of the analyzer 100. In one implementation, the analyzer 100 may comprise and/or be connectable to one or more sensor cartridge 143 (FIG. 12) having blood gas sensors 144, and/or one or more reagents cartridge 145.

In one embodiment, the analyzer 100 may comprise an optical input 112 and an optical receiver 114 positioned adjacent to the sample vessel 12, the optical input 112 positioned to emit the light beam 116 through the top 40, the bottom 42, and the microchannel 22, and the optical receiver 114 is positioned to receive at least a portion of the light beam 116 after the portion of the light beam 116 has passed through the top 40, the bottom 42, and the microchannel 22. In one embodiment, the optical input 112 may be connected to a first fiber optic bundle 118 that connects a light source 120 to the optical input 112. The optical receiver 114 may be connected to a second fiber optic bundle 119 that connects the optical receiver 114 to the absorbance spectrophotometer 102. The light source 120 may be one or more light emitting diode or a tungsten-halogen light, for example. In one embodiment, the light may be white light having wavelengths in a range from approximately 450-700 nanometers.

The absorbance spectrophotometer 102 may be configured to measure the intensity of light in a part of the spectrum, especially as transmitted or emitted by particular substances in the fluidic sample in the microchannel 22 of the sample vessel 12. The absorbance spectrophotometer 102 may be configured to measure how much a chemical substance absorbs light by measuring the intensity of light as a beam of light passes through the blood sample, or other fluidic sample. Each compound in the sample or solution absorbs or transmits light over a particular range of wavelengths.

The fluidic distribution system 104 may have an inlet 130 fluidly connectable to the first port 24, and an outlet 132 fluidly connectable to the second port 26 of the sample vessel 12 of the acoustophoretic device 10. The fluidic distribution system 104 may move one or more fluidic sample, such as a blank sample, or a blood sample, or a washing solution, through the inlet 130 through the first port 24 into the microchannel 22 of the sample vessel 12. In one embodiment, the fluidic distribution system 104 may flush the microchannel 22, expelling material within the microchannel 22 through the second port 26 of the sample vessel 12 and out of the outlet 132. The fluidic distribution system 104 may be a peri-pump, for instance, that may be operated automatically, manually, or a combination of automatically and manually. The fluidic distribution system 104 may be operated by the controller 106. In one embodiment, the inlet 130 may be the first tube 56 and the outlet 132 may be the second tube 58 and the inlet 52 may connect the first tube 56 to the first port 24 and the outlet 54 may connect the second tube 58 to the second port 26.

The controller 106 may be electrically connected to the piezo transducer 14 of the acoustophoretic device 10. The controller 106 may be configured to provide electrical signals to the piezo transducer 14, that when received by the piezo transducer 14 cause the piezo transducer 14 to emit ultrasonic acoustic waves at one or more frequency and/or range of frequencies, including at the resonant frequency of the monolithic structure of the acoustophoretic device 10 plus the fluidic sample.

As shown in FIG. 11, in one embodiment the controller 106 may have a first electrical contact 134 and a second electrical contact 136. The first electric contact 134 and the second electric contact 136 may be electrically connectable to the first terminal 90 and the second terminal 92, respectively, of the piezo transducer 14 of the acoustophoretic device 10 such that electrical potential may be provided to the piezo transducer 14.

The mount 108 may hold the acoustophoretic device 10 in place between the optical input 112 and the optical receiver 114 and may position the acoustophoretic device 10 to be operably connected to the fluidic distribution system 104 and the controller 106. The mount 108 may be configured to stabilize the acoustophoretic device 10 in position without applying a force that would significantly change the acoustic impedance of the monolithic structure of the acoustophoretic device 10. For example, the mount 108 may include one or more clamps that apply a clamping force at or below approximately twenty newtons (N).

In one embodiment, the analyzer 100 may further comprise a neon calibrator 122, a photo-feedback detector, one or more digital temperature sensors (not shown), and/or one or more thermal control element, such as Peltier elements (not shown).

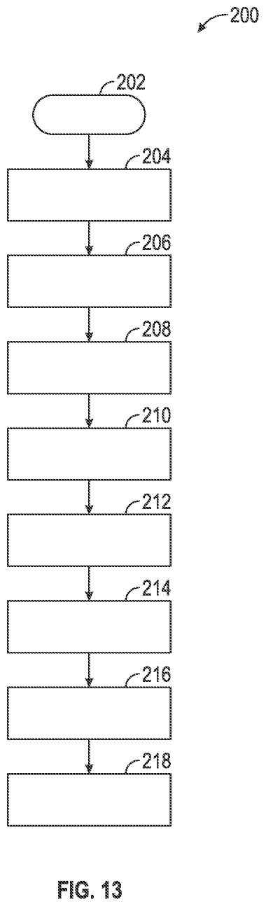

Referring now to FIGS. 13-14D, in one embodiment, a method 200 for analyzing blood may comprise a step 202 of obtaining or receiving a whole blood sample 60 containing red blood cells 62 and plasma 64 for analyzation.

In step 204, the whole blood sample 60 is positioned in the microchannel 22 of the acoustophoretic device 10 by pumping the whole blood sample 60 into the microchannel 22 through the inlet 130 and the first port 24 with the fluidic distribution system 104. The controller 106 may sense when the whole blood sample 60 is positioned in the microchannel 22 using the first electrode 32 and the second electrode 34 positioned on either side of the microchannel 22 to measure admittance magnitude and phase, for instance.

In step 206, the whole blood sample 60 in the microchannel 22 is pressurized. To pressurize the whole blood sample 60 in the microchannel 22, the controller 106 may be programmed to clamp or pinch the inlet 130 after the blood sample has been positioned in the microchannel 22 and reverse the fluidic distribution system 104 to pressurize the whole blood sample 60 in the microchannel 22. In one embodiment, the whole blood sample 60 may be pressurized to approximately 5 psi. In other embodiments, the whole blood sample 60 may be pressurized to a pressure between approximately 1 psi to approximately 10 psi.

In step 208, the controller 106 provides a first electrical signal to the piezo transducer 14 that causes the piezo transducer 14 to emit ultrasonic waves at a first frequency and/or range of frequencies to cause pre-enrichment of the red blood cells 62 and plasma 64. In particular, the first frequency may be produced with a first electrical voltage for a first predetermined period of time causing red blood cells 62 and plasma 64 in the whole blood sample 60 to begin to separate. For instance, the first frequency and/or range of frequencies may be between 950 kHz and 1100 kHz and the first electrical voltage may be between 50 volts and 100 volts. The first predetermined time period may be between 5 seconds and 15 seconds.

In step 210 (e.g., subsequent to step 208), the controller 106 provides a second electrical signal to the piezo transducer 14 that causes the piezo transducer 14 to emit ultrasonic waves at a second frequency and/or range of frequencies and at a second electrical voltage for a second predetermined period of time causing the red blood cells 62 and plasma 64 in the whole blood sample 60 to completely separate. By way of example, the second frequency and/or range of frequencies may be between 320 kHz and 500 kHz and the second electrical voltage may be between 70 volts and 100 volts. The second predetermined time period may be between 10 seconds and 25 seconds. In one exemplary embodiment, at least a portion of the plasma 64 is separated/moved or enriched toward a surface or outer edges of the microchannel 22 such that at least a portion of the plasma 64 (separated from the red blood cells 62) is located at anti-node region(s) proximate to the surface of the microchannel 22.

In step 212 (e.g., subsequent to steps 208 and 210), the controller 106 activates the light source 120 during the second predetermined time period (when the red blood cells 62 and plasma 64 are completely separated) to transmit the light beam 116 through the first fiber optic bundle 118 to the optical input 112 which directs at least a portion of the light beam 116 through the microchannel 22 at a predetermined position aligned with an anti-node region within the microchannel 22 being plasma 64 and substantially devoid of red blood cells 62. The optical receiver 114 receives the portion of the light beam 116 that was directed through the microchannel at the predetermined position and passes the received portion of the light beam 116 to the absorbance spectrophotometer 102 through the second fiber optic bundle 119 to take a plasma spectrum reading. In one exemplary embodiment, the predetermined position of the microchannel 22 at which the light beam 116 is directed may be anti-node region(s) located along outer edges of the microchannel 22 having plasma 64 and being substantially devoid of red blood cells 62 such that the plasma spectrum reading is taken or performed on the plasma 64 located at the anti-node region(s) along the outer edges of the microchannel 22. For example, a portion of the light beam 116 passing through the plasma 64 located at the anti-node region(s) along the outer edges/surfaces of the microchannel 22 is measured by the optical receiver (of the absorbance spectrophotometer) to determine optical absorbance of the plasma 64 (at the anti-node region(s) proximate the outer edges/surfaces of the microchannel 22), such as by comparing intensity of the light beam 116 received by the plasma 64 and outputted by the plasma 64 within a desired frequency range. The determined optical absorbance may then be used to determine the amount or concentration of plasma constituents of particular interest, such as bilirubin or free hemoglobin.

In a step 214, the microchannel is pressurized again.

In a step 216 after the absorbance spectrophotometer 102 has taken a plasma spectrum reading, the controller 106 provides a third electrical signal to the piezo transducer 14 that causes the piezo transducer 14 to emit ultrasonic waves at a third frequency and/or range of frequencies, including at the resonant frequency of the monolithic structure of the acoustophoretic device 10 plus the whole blood sample 60, and/or cause the piezo transducer 14 to elongate and contract thereby producing shear forces in the whole blood sample 60 in the microchannel 22 causing walls of the red blood cells 62 of the whole blood sample 60 to rupture or lyse producing a lysed blood sample 66.

In a step 218, the controller 106 activates the light source 120 to transmit the medium 116 to the optical input 112 which transmits at least a portion of the light beam 116 through the lysed blood sample 66 and to the optical receiver 114 which transmits the portion of the light beam 116 that passed through the lysed blood sample 66 to the absorbance spectrophotometer 102 to obtain a measurement of the lysed blood sample 66.

The method 200 may further comprise determining one or more oximetry parameters of the lysed blood sample based at least in part on a signal indicative of the portion of the light beam 116 received by the absorbance spectrophotometer 102.

It should be noted that the method 200 may be performed without pressurizing the whole blood sample 60 in steps 206 and 214. For example, in one embodiment, lysing the red blood cells 62 in step 216 may be performed directly after taking a plasma spectrum reading on the separated plasma 64 in step 212. It should further be noted that method 200 may be performed such that whole blood reconstitution does not occur, or in other words the red blood cells 62 and plasma 64 separated in step 208 or 210 are not capable of mixing to reconstitute as whole blood to produce a reconstituted whole blood sample. Thus, instead of producing reconstituted whole blood sample, the method 200 produces a lysed blood sample 66 in step 216 and as such method 200 may not include reversibility of the plasma separation steps 208 or 210. Further, it should be noted that the method 200 is performed within the microchannel 22 such that the steps of pre-enrichment of the red blood cells 62 and plasma 64 (step 208), complete separation of red blood cells 62 and plasma 64 in the whole blood sample 60 and measurement thereof (steps 210, 212), and lysing of the red blood cells 62 of the whole blood sample 60 and measurement thereof (step 216, 218) are all advantageously performed within the same, single (e.g., linear) microchannel 22 of the same sample vessel 12 thus advantageously avoiding the need for additional downstream channels or chambers in the sample vessel 12 or additional devices to transfer or collect the separated plasma or red blood cells.

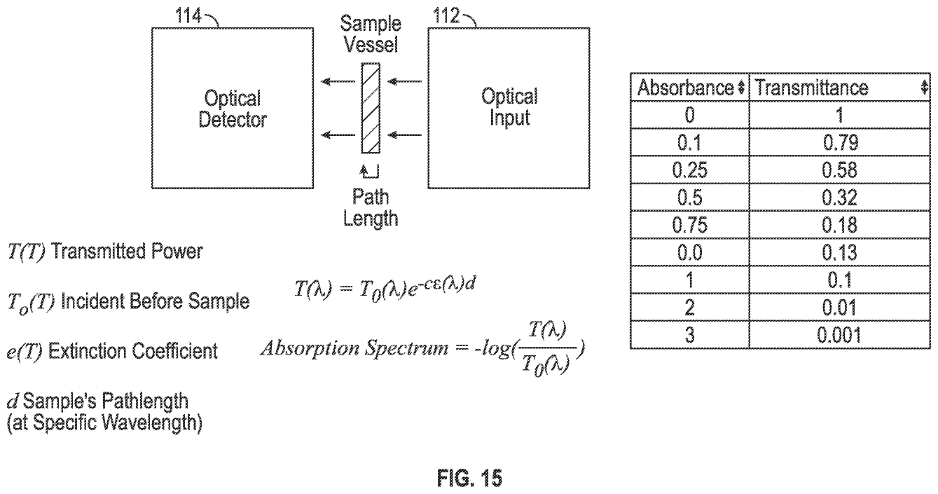

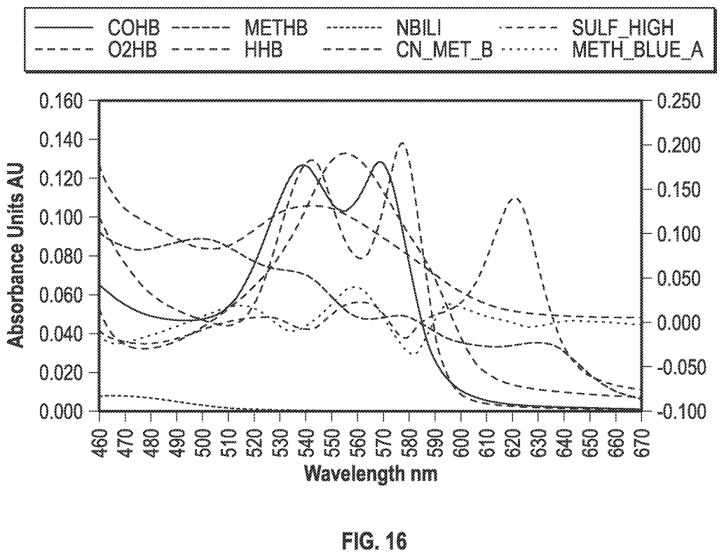

As shown in FIG. 15, an absorption spectrum may be calculated based on known calculations for absorption for liquid mediums. Further, as shown in FIG. 16, determining one or more oximetry parameters may further comprise analyzing spectral profile coefficients of hemoglobin forms, such as one or more of the following: Oxyhemoglobin (O2HB), Deoxyhemoglobin (HHB), Carboxyhemoglobin (COHB), Methemoglobin (METHB), and plasma Bilirubin (NBILI), and interfering substances Cyan Methemoglobin (CN_MET_B), Sulfhemoglobin (SULF_HIGH), and Methylene blue (METH_BLUE_A).

Determining one or more oximetry parameters may be based on measurement of spectrophotometric optical absorption, that is the absorption of light by components in the lysed blood sample 66.

Determining one or more oximetry parameters may comprise measuring at least total hemoglobin (THB) and one or more of hemoglobin fractions, such as the following: Oxyhemoglobin (O2HB), Deoxyhemoglobin (HHB), Carboxyhemoglobin (COHB) Methemoglobin (METHB).

In one embodiment, the method 200 may comprise inputting and evacuating a wash solution into the microchannel 22 of the sample vessel 12 before and/or after introducing the whole blood sample 60 into the microchannel 22. The method 200 may further comprise activating the piezo transducer 14 to produce acoustic waves and/or shear forces to agitate the wash solution in the microchannel 22. In one embodiment, the sample vessel 12 may be used, cleaned, and re-used. In one embodiment, the acoustophoretic device 10 may not be reusable, and may be replaced for each new whole blood sample 60. In such an embodiment, the acoustophoretic device 10 may be discarded after a single use.

The method 200 may further comprise calibrating the analyzer 100 with a fluidic sample. In one embodiment, the fluidic sample may be a test sample known as a “blank sample” that may be used to calibrate the analyzer 100. The blank sample may contain a dye solution, which may be used to measure scattering of the transmission of the medium 116.

In one embodiment, the whole blood sample 60 may be approximately twelve microliters in volume. The whole blood sample 60 typically comprises plasma 64 and red blood cells 62 (which may comprise 45%-60% of the blood sample) and possibly lipids.

In one embodiment, the whole blood sample 60 is held at a consistent temperature. In one embodiment, the temperature of the whole blood sample 60 is thirty-seven degrees Celsius (37° C.) plus or minus approximately 0.3 degree. In one embodiment, the temperature of the whole blood sample 60 is less than forty degrees Celsius (40° C.) to avoid damage to the whole blood sample 60. In one embodiment, the whole blood sample 60 is held at a substantially consistent temperature utilizing one or more temperature sensor (not shown) and/or one or more thermal control element (not shown).

An example of the analyzer 100 and the acoustophoretic device 10 in use will now be described. In one example, the sample vessel 12 may be made of glass and may have a length-to-width aspect ratio in a range of about 1.4 to about 1.9, and the microchannel 22 may have a height-to-width aspect ratio of about 0.05 (for example, having a height of about 100 micrometers and a width of about two millimeters). The sample vessel 12 may be inserted in a path that the medium 116 will travel between the optical input 112 and the optical receiver 114 of the absorbance spectrophotometer 102. It should be understood that the analyzer 100 may be provided with various instruments including mirrors and/or waveguides to direct the medium through the path. The fluidic distribution system 104 may insert the whole blood sample 60 into the microchannel 22 of the sample vessel 12.

The controller 106 may be electrically connected to the piezo transducer(s) 14 of the sample vessel 12, and may provide electrical signals to the piezo transducer(s) 14 to cause the piezo transducer 14 to emit first ultrasonic sound waves through a range of frequencies from approximately 950 kHz to approximately 1100 kHz in steps of approximately 1 kHz. The range of frequencies may be transmitted within a time period of approximately 5 seconds to approximately 15 seconds. This causes the red blood cells 62 to begin to separate from the plasma 64 in the whole blood sample 60 as shown in FIG. 14B. For the sake of clarity, the red blood cells 62 are shown as circles and the plasma 64 is represented by lines in FIGS. 14A-14D.

The controller 106 may then provide electrical signals to the piezo transducer(s) 14 to cause the piezo transducer 14 to emit second ultrasonic sound waves through a range of frequencies from approximately 320 kHz to approximately 500 kHz in steps of approximately 1 kHz. The range of frequencies may be transmitted within a time period of approximately 10 seconds to approximately 25 seconds. Transmission of the second ultrasonic sound waves through the whole blood sample 60 completes the separation of the red blood cells 62 from the plasma 64 as shown in FIG. 14C.

Standing waves produced by the second ultrasonic sound waves cause the red blood cells 62 and the plasma 64 to move to known portions of the microchannel 22 as illustrated in FIG. 14C. Anti-node regions 67 (only anti-node regions 67a and 67b are numbered in the Figures) and node regions 68 (only one of which is numbered in the Figures) between the anti-node regions 67 form the known portions of the microchannel 22. The separated plasma 64 is enriched in (e.g., separated/moved to) the anti-node regions 67 within the microchannel 22 which are substantially devoid of red blood cells 62, while the red blood cells 62 move to node regions 68. As illustrated in FIG. 14C, a plurality of interleaved node regions 68 and anti-node regions 67 may form along outer edges of the microchannel 22 (such as exemplary anti-node region 67a) and a plurality of anti-node regions 67 may form along a central axis of the microchannel 22 (such as exemplary anti-node region 67b). The outer edges of the microchannel 22 may also be referred to as sides, walls or surfaces of the microchannel 22 such that anti-node region(s) 67a is formed proximate the sides, walls or surfaces of the microchannel 22, while anti-node region(s) 67b is formed at a center of the microchannel 22. Node regions 68 may form in the remaining spaces of the microchannel 22. A desired pattern of anti-node regions 67 and node regions 68 may be calculated using the techniques described above.

The controller 106 may then cause the light source 120 to emit the light beam 116 directed by the optical input 112 through a predetermined portion of the microchannel 22 (i.e., in one of the anti-node regions 67), where plasma 64 has separated from the red blood cells 62 of the whole blood sample 60. At least a portion of the light beam 116 passes through the plasma 64 and is received by the optical receiver 114 and directed to the absorbance spectrophotometer 102 to obtain a plasma measurement. Measurements on plasma can also be made using absorbance spectroscopy. A patient blood sample (i.e., the whole blood sample) includes plasma and red blood cells. Red blood cells (RBCs) are separated from the plasma as discussed above, to physically separate the plasma to measure plasma constituents of particular interest, such as bilirubin or free hemoglobin. The RBCs contain the forms of hemoglobin: Oxyhemoglobin, Deoxyhemoglobin, Carboxyhemoglobin and Methemoglobin. In some cases, free hemoglobin can be found in the plasma. Free hemoglobin can be created by improper phlebotomy (blood sample draw), or patient physiological condition such as hemolytic anemia.

In one exemplary embodiment, the predetermined portion of the microchannel 22 through which the light beam 116 passes is anti-node region(s) 67a located along the outer edges of the microchannel 22. In this embodiment, the controller 106 is configured to cause the light source 120 to emit the light beam 116 for passing through the anti-node region(s) 67a at the sides of the microchannel 22 where plasma has separated. Thus, at least a portion of the light beam 116 passes through the plasma 64 at the anti-node regions 67a formed proximate the surface of the microchannel 22 and is received by the optical receiver 114 of the absorbance spectrophotometer 102 to obtain a plasma measurement of plasma constituents at the anti-node regions 67a (i.e., at the sides of the microchannel 22). The optical receiver 114 and/or absorbance spectrophotometer 102 may be aligned with sides of the microchannel 22 to receive the portion of the light beam 116 passing through the plasma 64 at the anti-node regions 67a and to perform optical detection of the plasma formed along the sides of the microchannel 22 (i.e., anti-node region(s) 67a) to detect a constituent(s) or analyte(s) through measurement of the plasma at the anti-node region(s) 67a. Because there is less optical interference at the sides of the microchannel 22 (compared to the center of the microchannel 22), measurement or detection of plasma constitutes at anti-node region(s) 67a along the sides of the microchannel 22 advantageously results in improved accuracy of plasma measurement as compared to measurement at the anti-node region(s) 67b along a central axis of the microchannel 22.

Once the plasma measurement has been obtained, the controller 106 may provide electrical signals to the piezo transducer(s) 14 to cause the piezo transducer 14 to emit third ultrasonic sound waves through a range of frequencies from approximately 330 kHz to approximately 370 kHz in steps of approximately 1 kHz. The range of frequencies may be transmitted within a time period of approximately two seconds.

The frequency, intensity, and duration of the third ultrasonic sound waves lyse the red blood cells 62 within the whole blood sample 60 causing the red blood cells 62 to release hemoglobin from within the red blood cell 62, as shown in FIG. 14D. The frequency range of the third ultrasonic sound waves includes the resonant frequency for the monolithic structure of the acoustophoretic device 10 with the whole blood sample 60, thereby causing cavitation in the whole blood sample 60, which ruptures cell walls of the red blood cells 62 in the whole blood sample 60. Additionally, or alternatively, the controller 106 may cause the one or more processors 140 to pass signals to the piezo transducer(s) 14 that cause the piezo transducer(s) 14 to elongate and contract, thereby producing shear forces in the whole blood sample 60 in the microchannel 22, which rupture the cell walls of the red blood cells 62 in the whole blood sample 60 producing the lysed blood sample 66. In the lysed blood sample 66, the lysed red blood cells are shown as dots.

A majority (more than 50%) of the cell walls of the red blood cells 62 may be ruptured.

After the lysed blood sample 66 has been produced, the controller 106 may activate the light source 120 of the analyzer 100 to transmit the medium 116, such as light, through the sample vessel 12 into the lysed blood sample 66. The optical receiver 114 may receive at least portions of the medium 116 that exits the lysed blood sample 66 and the sample vessel 12. The optical receiver 114 may include one or more photodiodes, for example, for generating an electrical signal due to reception of the medium 116.

The analyzer 100, or the one or more computer processors 140, may determine one or more analytes present in the lysed blood sample based at least in part on a signal indicative of the light received by the optical receiver 114 of the absorbance spectrophotometer 102. The analyzer 100, or one or more computer processors, may further analyze spectral profile coefficients of hemoglobin forms, such as one or more of the following: carboxyhemoglobin (COHB), oxyhemoglobin (O2HB), methemoglobin (METHB), deoxyhemoglobin (HHB), neonatal Bilirubin (NBILI), Cyan Methemoglobin (CN_MET_B), Sulfhemoglobin (SULF_HIGH), Methylene blue dye (METH_BLUE_A).

The analyzer 100, or the one or more computer processors 140, may measure total hemoglobin (THB) and/or one or more of hemoglobin fractions, such as the following: oxyhemoglobin (O2HB), methemoglobin (METHB), deoxyhemoglobin (HHB), carboxyhemoglobin (COHB).

The analyzer 100, or the one or more computer processors 140, may output the result of the analyses. The output may be shown on one or more display. The output may be used to determine treatment of the patient.

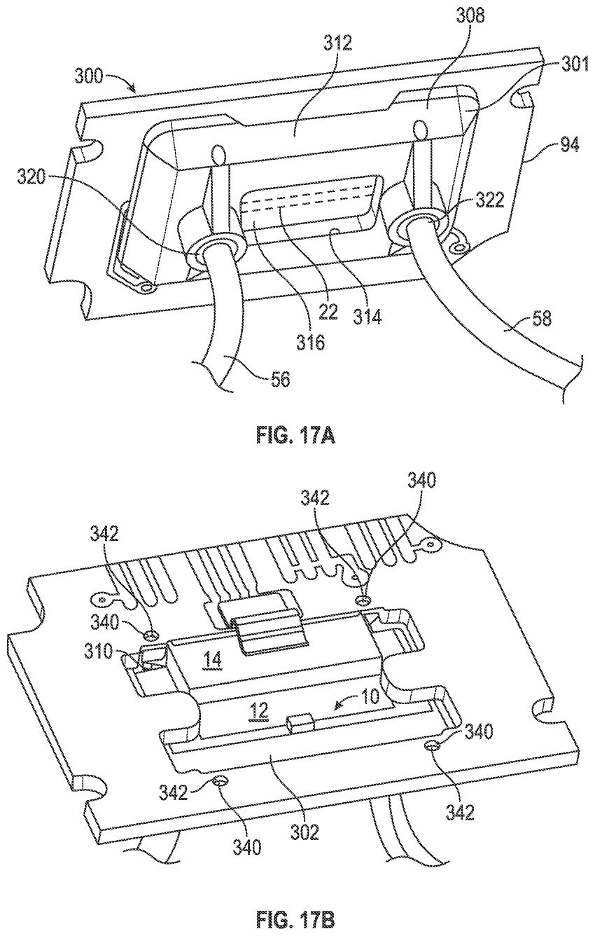

Shown in FIGS. 17A and 17B is an assembly 300 constructed in accordance with the present invention for use within the analyzer 100. The assembly 300 is provided with the acoustophoresis device 10, the PCB 94, and a flowcell holder 301. The flowcell holder 301 is configured to be mounted to the PCB 94 so as to define a cavity 302 sized and dimensioned to receive the acoustophoresis device 10. When the acoustophoresis device 10 is positioned within the cavity 302, the flowcell holder 301 and the PCB 94 press against the acoustophoresis device 10 to hold the acoustophoresis device 10 securely within the cavity 302 so as to prevent movement of the acoustophoresis device 10 relative to the flowcell holder 301 and the PCB 94. In this regard, the flowcell holder 301 is provided with a body 308 having a first surface 310 configured to matingly engage at least a portion of the acoustophoresis device 10 and a second surface 312 opposite the first surface 310. The body 308 also includes an inner wall 314 extending between the first surface 310 and the second surface 312 so as to define an opening 316 extending through the body 308. The opening 316 is positioned within the body 308 such that upon placement of the acoustophoresis device 10 within the cavity 302, the opening 316 is aligned with the microchannel 22. Aligning the opening 316 with the microchannel 22 permits a light beam to pass through the microchannel 22 and the opening 316 so that a measurement, e.g., an absorbance measurement can be taken of content, e.g., a sample, within the microchannel 22. The body 308 of the flowcell holder 301 also includes an inlet aperture 320 and an outlet aperture 322 extending from the first surface 310 to the second surface 312. The inlet aperture 320 securely receives the first tube 56 and the outlet aperture 322 securely receives the second tube 58. For example, the inlet aperture 320 may have an inner diameter that is less than an outer diameter of the first tube 56 so as to securely receive the first tube 56. The outlet aperture 312 and the second tube 58 can be constructed similarly as the inlet aperture 320 and the first tube 56. In this embodiment, the inlet and outlet tube fittings 52 and 54 are optional. In some embodiments, the body 308 of the flowcell holder 301 presses the first tube 56 and the second tube 58 against the sample vessel 12 adjacent to the first port 24 and the second port 26 so as to form and maintain a fluid connection between the first tube 56 and the first port 24, and the second tube 58 and the second port 26.

The flowcell holder 301 can be secured to the PCB 94 so as to hold the acoustophoresis device 10 against the PCB 94 without soldering. For example, the PCB 94 and the body 308 can be provided with a series of aligned holes 340 adapted to receive fasteners 342 connecting the flowcell holder 301 to the PCB 94. In the example shown, holes within the body 308 receiving the fasteners 342 are not shown. In the example shown, the PCB 94 and the body 308 are provided with four aligned holes 340 adapted to receive and/or receiving the fasteners 342. The fasteners 342 can be screws, for example.

In this example, mounting pads on the sample vessel 12 are pressed against mounting pads on the PCB 94 to form an electrical connection therebetween. The assembly 300 can be a component of the analyzer 100 and used for performing absorbance measurements on a blood sample, as described above.

The following is a number list of non-limiting illustrative embodiments of the inventive concept disclosed herein:

1. A fluid analyzation device, comprising:
a sample vessel having an outer surface, a microchannel within the confines of the outer surface, a first port extending through the outer surface to the microchannel, and a second port extending through the outer surface to the microchannel, such that a blood sample is insertable through the first port into the microchannel; and
a piezo transducer bonded to the outer surface of the sample vessel to form a monolithic structure,
a controller configured to send signals to the piezo transducer at a separation frequency and a lysis frequency, the separation frequency configured to cause the piezo transducer to impart separation acoustic waves into the sample vessel sufficient to separate red blood cells and plasma of the blood sample without rupturing the red blood cells, and the lysis frequency configured to cause the piezo transducer to impart lysis acoustic waves into the sample vessel sufficient to induce shear forces within the microchannel configured to induce cavitation in the blood sample such that cell walls of the red blood cells in the blood sample are ruptured and release hemoglobin from within the red blood cells.

2. The fluid analyzation device of illustrative embodiment 1, wherein the sample vessel is constructed of glass.

3. The fluid analyzation device of any one of the preceding illustrative embodiments, wherein the separation frequency comprises a pre-enrichment frequency and an enrichment frequency, wherein the controller is configured to send signals to the piezo transducer at the pre-enrichment frequency between substantially 950 kHz and substantially 1100 kHz, and wherein the controller is further configured to send signals to the piezo transducer at the enrichment frequency sufficient to cause the piezo transducer to impart enrichment acoustic waves into the sample vessel without rupturing the red blood cells.

4. The fluid analyzation device of any one of the preceding illustrative embodiments, wherein the enrichment frequency is a frequency between substantially 320 kHz and substantially 500 kHz.

5. The fluid analyzation device of any one of the preceding illustrative embodiments, wherein the lysis frequency is a frequency between substantially 300 kHz and substantially 370 kHz.

6. The fluid analyzation device of any one of the preceding illustrative embodiments, wherein the lysis frequency is a range of frequencies between substantially 300 kHz and substantially 370 kHz and the piezo transducer is configured to sweep the range of frequencies between substantially 300 kHz and substantially 370 kHz.

7. The fluid analyzation device of any one of the preceding illustrative embodiments, wherein the outer surface is a first outer surface having a mounting area, the mounting area having a first shape, and wherein the piezo transducer has a second outer surface having a second shape corresponding to the first shape, the second outer surface of the piezo transducer bonded to the mounting area.

8. The fluid analyzation device of any one of the preceding illustrative embodiments, wherein the piezo transducer matingly engages the outer surface of the sample vessel.

9. The fluid analyzation device of any one of the preceding illustrative embodiments, wherein the acoustic waves are configured to cause the plasma to separate to an anti-node region located proximate to the outer surface of the microchannel, and wherein an optical detector is positioned to receive light passing through the plasma located at the anti-node region and is configured to detect plasma constituents based on measurement of the plasma located at the anti-node region proximate the outer surface.

10. A fluid analyzer, comprising:
a fluid analyzation device, comprising:
a sample vessel having an outer surface, a microchannel within the confines of the outer surface, a first port extending through the outer surface to the microchannel, and a second port extending through the outer surface to the microchannel, such that a blood sample is insertable through the first port into the microchannel; and
a piezo transducer bonded to the outer surface of the sample vessel to form a monolithic structure, the piezo transducer configured to generate first ultrasonic acoustic waves having a first frequency, second ultrasonic acoustic waves having a second frequency, and third ultrasonic acoustic waves having a third frequency, the first ultrasonic acoustic waves configured to begin separation of red blood cells and plasma in the blood sample in the microchannel, the second ultrasonic acoustic waves configured to substantially complete separation of the red blood cells and plasma in the blood sample in the microchannel, and the third ultrasonic acoustic waves configured to vibrate the sample vessel such that shear forces are induced within the microchannel, the third ultrasonic acoustic waves and the shear forces configured to induce cavitation in the blood sample in the microchannel such that cell walls of the red blood cells in the blood sample are ruptured and release hemoglobin from within the red blood cells;
an absorbance spectrophotometer comprising an optical transmitter and an optical receiver positioned adjacent to the sample vessel, the optical transmitter positioned to emit a light medium through the microchannel, the optical receiver positioned to receive at least a portion of the light medium after the portion of the light medium has passed through the microchannel;
a fluidic distribution system having an outlet connected to the first port, and an inlet connected to the second port; and
a controller electrically connected to the piezo transducer, the controller configured to send electrical signals to the piezo transducer that when received by the piezo transducer cause the piezo transducer to emit the first ultrasonic acoustic waves, the second ultrasonic acoustic waves, and the third ultrasonic acoustic waves.

11. The fluid analyzer of illustrative embodiment 10, wherein the sample vessel is constructed of glass.

12. The fluid analyzer of any one of illustrative embodiments 10-11, wherein the first frequency is a frequency between substantially 950 kHz and substantially 1100 kHz.

13. The fluid analyzer of any one of illustrative embodiments 10-12, wherein the second frequency is a frequency between substantially 320 kHz and substantially 500 kHz.

14. The fluid analyzer of any one of illustrative embodiments 10-13, wherein the third frequency is a frequency between substantially 300 kHz and substantially 370 kHz.

15. The fluid analyzer of one of illustrative embodiments 10-14, wherein the third frequency is a range of frequencies between substantially 300 kHz and substantially 370 kHz and the piezo transducer is configured to sweep the range of frequencies between substantially 300 kHz and substantially 370 kHz.

16. The fluid analyzer of any one of illustrative embodiments 10-15, wherein the outer surface of the sample vessel is a first outer surface having a mounting area, the mounting area having a first shape, and wherein the piezo transducer has a second outer surface having a second shape corresponding to the first shape, the second outer surface of the piezo transducer bonded to the mounting area.

17. The fluid analyzer of any one of illustrative embodiments 10-16, wherein the piezo transducer matingly engages the outer surface of the sample vessel.

18. The fluid analyzer of any one of the preceding illustrative embodiments, wherein the second ultrasonic acoustic waves are configured to cause the plasma to separate to an anti-node region located proximate to the outer surface of the microchannel, and wherein the absorbance spectrophotometer is configured to perform a measurement on the plasma located at the anti-node region proximate the outer surface to determine plasma analytes.

19. A blood analyzation method, comprising steps of:
passing a whole blood sample having red blood cells and plasma into a microchannel of a sample vessel;
separating the red blood cells from the plasma within the microchannel;
taking a first absorbance spectroscopy reading of the plasma separated from the red blood cells;
lysing the red blood cells within the microchannel to provide a lysed blood sample; and
taking a second absorbance spectroscopy reading of the lysed blood sample within the microchannel.

20. The method of illustrative embodiment any one of the preceding illustrative embodiments, wherein separating the red blood cells from the plasma within the microchannel is defined further as inducing first acoustic waves within the microchannel at a sufficient frequency and duration to begin separation of the red blood cells from the plasma and inducing second acoustic waves within the microchannel at a sufficient frequency and duration to substantially complete separation of the red blood cells and the plasma.

21. The method of any one of the preceding illustrative embodiments, wherein the sufficient frequency of the first acoustic waves is a frequency between substantially 950 kHz and substantially 1100 kHz and the duration is a time period between substantially five seconds and substantially fifteen seconds.

22. The method of any one of the preceding illustrative embodiments, wherein the sufficient frequency of the second acoustic waves is a frequency between substantially 320 kHz and substantially 500 kHz and the duration is a time period between substantially ten seconds and substantially twenty-five seconds.

23. The method of any one of the preceding illustrative embodiments, wherein lysing the red blood cells within the microchannel to provide a lysed blood sample is defined further as inducing third acoustic waves within the microchannel at a sufficient frequency and duration to induce shear forces within the microchannel configured to induce cavitation in the blood sample such that cell walls of the red blood cells are lysed and release hemoglobin from the red blood cells.

24. The method of any one of the preceding illustrative embodiments, wherein the sufficient frequency of the third acoustic waves is a frequency between substantially 320 kHz and substantially 370 kHz and the duration is a time period between substantially two seconds and substantially twenty seconds.

25. The method of any one of the preceding illustrative embodiments, wherein the separating step enriches the plasma to an anti-node region proximate to a surface of the microchannel, and wherein the step of taking the first absorbance spectroscopy reading is performed on the plasma located at the anti-node region proximate the surface of the microchannel.

### Conclusion

Conventionally, blood analysis was not available at the point-of-care of patients or was time consuming and expensive. In accordance with the present disclosure, the analyzer 100 is disclosed which provides improved accuracy and precision of measured parameters of a blood sample within a desired time-to-result at the point of care of a patient, and that is more easily manufactured and with less cost. The controller 106 of the analyzer 100 sends predetermined signals to the piezo transducer 14 to impart acoustic waves into the sample vessel 12 to first separate plasma 64 from other constituents of whole blood such as red blood cells 62 to obtain a plasma measurement, then lyse the red blood cells 62 in the sample vessel 12 by means of ultrasonic acoustic waves, pressure, cavitation, fluid movement, and/or shear forces, generated in the sample vessel 12 by a single piezo transducer 14 driven at one or more particular excitation frequency, or range of frequencies

The foregoing description provides illustration and description, but is not intended to be exhaustive or to limit the inventive concepts to the precise form disclosed. Modifications and variations are possible in light of the above teachings or may be acquired from practice of the methodologies set forth in the present disclosure.

Even though particular combinations of features and steps are recited in the claims and/or disclosed in the specification, these combinations are not intended to limit the disclosure. In fact, many of these features and steps may be combined in ways not specifically recited in the claims and/or disclosed in the specification. Although each dependent claim listed below may directly depend on only one other claim, the disclosure includes each dependent claim in combination with every other claim in the claim set.

No element, act, or instruction used in the present application should be construed as critical or essential to the invention unless explicitly described as such outside of the preferred embodiment. Further, the phrase “based on” is intended to mean “based, at least in part, on” unless explicitly stated otherwise.

What is claimed is:

1. A fluid analyzation device, comprising:
a sample vessel having an outer surface, a microchannel within the confines of the outer surface, a first port extending through the outer surface to the microchannel, and a second port extending through the outer surface to the microchannel, such that a blood sample is insertable through the first port into the microchannel; and
a piezo transducer bonded to the outer surface of the sample vessel to form a monolithic structure,
a controller configured to send signals to the piezo transducer at a separation frequency and a lysis frequency, the separation frequency configured to cause the piezo transducer to impart separation acoustic waves into the sample vessel sufficient to separate red blood cells and plasma of the blood sample without rupturing the red blood cells, and the lysis frequency configured to cause the piezo transducer to impart lysis acoustic waves into the sample vessel sufficient to induce shear forces within the microchannel configured to induce cavitation in the blood sample such that cell walls of the red blood cells in the blood sample are ruptured and release hemoglobin from within the red blood cells.

2. The fluid analyzation device of claim 1, wherein the sample vessel is constructed of glass.

3. The fluid analyzation device of claim 1, wherein the separation frequency comprises a pre-enrichment frequency and an enrichment frequency, wherein the controller is configured to send signals to the piezo transducer at the pre-enrichment frequency between substantially 950 kHz and substantially 1100 kHz, and wherein the controller is further configured to send signals to the piezo transducer at the enrichment frequency sufficient to cause the piezo transducer to impart enrichment acoustic waves into the sample vessel without rupturing the red blood cells.

4. The fluid analyzation device of claim 3, wherein the enrichment frequency is a frequency between substantially 320 kHz and substantially 500 kHz.

5. The fluid analyzation device of claim 1, wherein the lysis frequency is a frequency between substantially 300 kHz and substantially 370 kHz.

6. The fluid analyzation device of claim 5, wherein the lysis frequency is a range of frequencies between substantially 300 kHz and substantially 370 kHz and the piezo transducer is configured to sweep the range of frequencies between substantially 300 kHz and substantially 370 kHz.

7. The fluid analyzation device of claim 1, wherein the outer surface is a first outer surface having a mounting area, the mounting area having a first shape, and wherein the piezo transducer has a second outer surface having a second shape corresponding to the first shape, the second outer surface of the piezo transducer bonded to the mounting area.

8. The fluid analyzation device of claim 1, wherein the piezo transducer matingly engages the outer surface of the sample vessel.

9. The fluid analyzation device of claim 1, wherein the separation acoustic waves are configured to cause the plasma to separate to an anti-node region located proximate to the outer surface of the microchannel, and wherein an optical detector is positioned to receive light passing through the plasma located at the anti-node region and is configured to detect plasma constituents based on measurement of the plasma located at the anti-node region proximate the outer surface.

10. A fluid analyzer, comprising:
a fluid analyzation device, comprising:
a sample vessel having an outer surface, a microchannel within the confines of the outer surface, a first port extending through the outer surface to the microchannel, and a second port extending through the outer surface to the microchannel, such that a blood sample is insertable through the first port into the microchannel; and
a piezo transducer bonded to the outer surface of the sample vessel to form a monolithic structure, the piezo transducer configured to generate first ultrasonic acoustic waves having a first frequency, second ultrasonic acoustic waves having a second frequency, and third ultrasonic acoustic waves having a third frequency, the first ultrasonic acoustic waves configured to begin separation of red blood cells and plasma in the blood sample in the microchannel, the second ultrasonic acoustic waves configured to substantially complete separation of the red blood cells and plasma in the blood sample in the microchannel, and the third ultrasonic acoustic waves configured to vibrate the sample vessel such that shear forces are induced within the microchannel, the third ultrasonic acoustic waves and the shear forces configured to induce cavitation in the blood sample in the microchannel such that cell walls of the red blood cells in the blood sample are ruptured and release hemoglobin from within the red blood cells;
an absorbance spectrophotometer comprising an optical transmitter and an optical receiver positioned adjacent to the sample vessel, the optical transmitter positioned to emit a light medium through the microchannel, the optical receiver positioned to receive at least a portion of the light medium after the portion of the light medium has passed through the microchannel;
a fluidic distribution system having an outlet connected to the first port, and an inlet connected to the second port; and
a controller electrically connected to the piezo transducer, the controller configured to send electrical signals to the piezo transducer that when received by the piezo transducer cause the piezo transducer to emit the first ultrasonic acoustic waves, the second ultrasonic acoustic waves, and the third ultrasonic acoustic waves.

11. The fluid analyzer of claim 10, wherein the sample vessel is constructed of glass.

12. The fluid analyzer of claim 10, wherein the first frequency is a frequency between substantially 950 kHz and substantially 1100 kHz.

13. The fluid analyzer of claim 10, wherein the second frequency is a frequency between substantially 320 kHz and substantially 500 kHz.

14. The fluid analyzer of claim 10, wherein the third frequency is a frequency between substantially 300 kHz and substantially 370 kHz.

15. The fluid analyzer of claim 14, wherein the third frequency is a range of frequencies between substantially 300 kHz and substantially 370 kHz and the piezo transducer is configured to sweep the range of frequencies between substantially 300 kHz and substantially 370 kHz.

16. The fluid analyzer of claim 10, wherein the outer surface of the sample vessel is a first outer surface having a mounting area, the mounting area having a first shape, and wherein the piezo transducer has a second outer surface having a second shape corresponding to the first shape, the second outer surface of the piezo transducer bonded to the mounting area.

17. The fluid analyzer of claim 10, wherein the piezo transducer matingly engages the outer surface of the sample vessel.

18. The fluid analyzer of claim 10, wherein the second ultrasonic acoustic waves are configured to cause the plasma to separate to an anti-node region located proximate to the outer surface of the microchannel, and wherein the absorbance spectrophotometer is configured to perform a measurement on the plasma located at the anti-node region proximate the outer surface to determine plasma analytes.

19. A blood analyzation method, comprising steps of:
passing a whole blood sample having red blood cells and plasma into a microchannel of a sample vessel;
separating the red blood cells from the plasma within the microchannel;
taking a first absorbance spectroscopy reading of the plasma separated from the red blood cells;
lysing the red blood cells within the microchannel to provide a lysed blood sample; and
taking a second absorbance spectroscopy reading of the lysed blood sample within the microchannel.

20. The method of claim 19, wherein separating the red blood cells from the plasma within the microchannel is defined further as inducing first acoustic waves within the microchannel at a sufficient frequency and duration to begin separation of the red blood cells from the plasma and inducing second acoustic waves within the microchannel at a sufficient frequency and duration to substantially complete separation of the red blood cells and the plasma.

21. The method of claim 20, wherein the sufficient frequency of the first acoustic waves is a frequency between substantially 950 kHz and substantially 1100 kHz and the duration is a time period between substantially five seconds and substantially fifteen seconds.

22. The method of claim 20, wherein the sufficient frequency of the second acoustic waves is a frequency between substantially 320 kHz and substantially 500 kHz and the duration is a time period between substantially ten seconds and substantially twenty-five seconds.

23. The method of claim 19, wherein lysing the red blood cells within the microchannel to provide a lysed blood sample is defined further as inducing third acoustic waves within the microchannel at a sufficient frequency and duration to induce shear forces within the microchannel configured to induce cavitation in the blood sample such that cell walls of the red blood cells are lysed and release hemoglobin from the red blood cells.

24. The method of claim 23, wherein the sufficient frequency of the third acoustic waves is a frequency between substantially 320 kHz and substantially 370 kHz and the duration is a time period between substantially two seconds and substantially twenty seconds.

25. The method of claim 19, wherein the separating step enriches the plasma to an anti-node region proximate to a surface of the microchannel, and wherein the step of taking the first absorbance spectroscopy reading is performed on the plasma located at the anti-node region proximate the surface of the microchannel.
